(19) United States
(12) Patent Application Publication
(10) Pub. No.: US 2014/0166016 A1
(43) Pub. Date: Jun. 19, 2014

(54) Respiratory Mask

(71) Applicant: HSINER CO., LTD., Taichung City (TW)

(72) Inventor: Eric CHANG, Taichung City (TW)

(73) Assignee: HSINER CO., LTD., Taichung City (TW)

(21) Appl. No.: 13/715,001

(22) Filed: Dec. 14, 2012

(51) Int. Cl. A61M 16/06; A61M 16/00

(52) U.S. Cl. CPC A61M 16/06; A61M 16/0057
USPC 128/205.25

(57) ABSTRACT

A respiratory mask includes a cup-shaped body having an annular flange, and a hollow face-contact body having an annular connecting end portion embedding the annular flange. The annular flange has opposite first and second annular surfaces, a plurality of angularly spaced-apart through holes, and an outer annular peripheral edge interconnecting the first and second annular surfaces. The annular connecting end portion includes outer and inner annular plates abutting against the first and second annular surfaces, respectively, a plurality of connecting ribs respectively filling the through holes and interconnecting the inner and outer annular plates, and an annular rim surrounding the outer annular peripheral edge.

### BACKGROUND OF THE INVENTION

1. Field of the Invention

This invention relates to a respiratory mask, more particularly to a respiratory mask having a cup-shaped body and a face-contact body.

2. Description of the Related Art

For treating a sleep apnea patient, a respiratory mask is usually used to cover the nose or the nose and mouth of the patient for assisting the patient's respiration or to administer specific treatment gas so that physiological functions of the patient can be maintained.

Referring to FIG. 1, a respiratory mask, as disclosed in US Patent Publication No. US2010/0282264A1, comprises a face-contact body 1, a cup-shaped body 2, and an air supply tube 3 connected to the cup-shaped body 2. The face-contact body 1 is made of silicone and is hollow, and has an annular end face 101 and an annular engaging groove 102 extending inwardly from the annular end face 101 and having a substantially U-shaped cross-section. The cup-shaped body 2 is made of rigid plastic and has an annular engaging portion 201 engaged to the engaging groove 102. The annular engaging portion 201 also has a substantially U-shaped cross-section.

Although the cup-shaped body 2 can be made before forming the face-contact body 1 in a mold so that the engaging portion 201 and the engaging groove 102 can be interengaged, because there is no other connection between the engaging portion 201 and the engaging groove 102, when high pressure air is supplied into the respiratory mask, a junction between the engaging portion 201 and the engaging groove 102 is easily deformed that may result in air leakage. Further, during cleaning of the respiratory mask, the face-contact body 1 and the cup-shaped body 2 are liable to separate. Once separated, reassembly of the face-contact body 1 and the cup-shaped body 2 is relatively difficult.

### SUMMARY OF THE INVENTION

Therefore, an object of the present invention is to provide a respiratory mask that is capable of overcoming the aforesaid drawbacks of the prior art.

According to this invention, a respiratory mask comprises a cup-shaped body and a hollow face-contact body. The cup-shaped body has an open end and an annular flange extending outwardly from the open end. The annular flange has a first annular surface, a second annular surface opposite to the first annular surface, a plurality of angularly spaced-apart through holes extending through the first and second annular surfaces, and an outer annular peripheral edge interconnecting outer peripheries of the first and second annular surfaces and surrounding the through holes. The hollow face-contact body has a surrounding wall and an annular connecting end portion connected integrally as one piece with the surrounding wall. The annular connecting end portion embeds the annular flange and includes an outer annular plate abutting against the first annular surface of the annular flange, an inner annular plate abutting against the second annular surface of the annular flange, a plurality of connecting ribs respectively filling the through holes and interconnecting the inner and outer annular plates, and an annular rim surrounding the outer annular peripheral edge of the annular flange and interconnecting outer ends of the inner and outer annular plates.

### BRIEF DESCRIPTION OF THE DRAWINGS

Other features and advantages of the present invention will become apparent in the following detailed description of the preferred embodiment with reference to the accompanying drawings, of which:

FIG. 1 is a sectional view of a respiratory mask disclosed in Taiwanese Patent Publication No. 201034709;

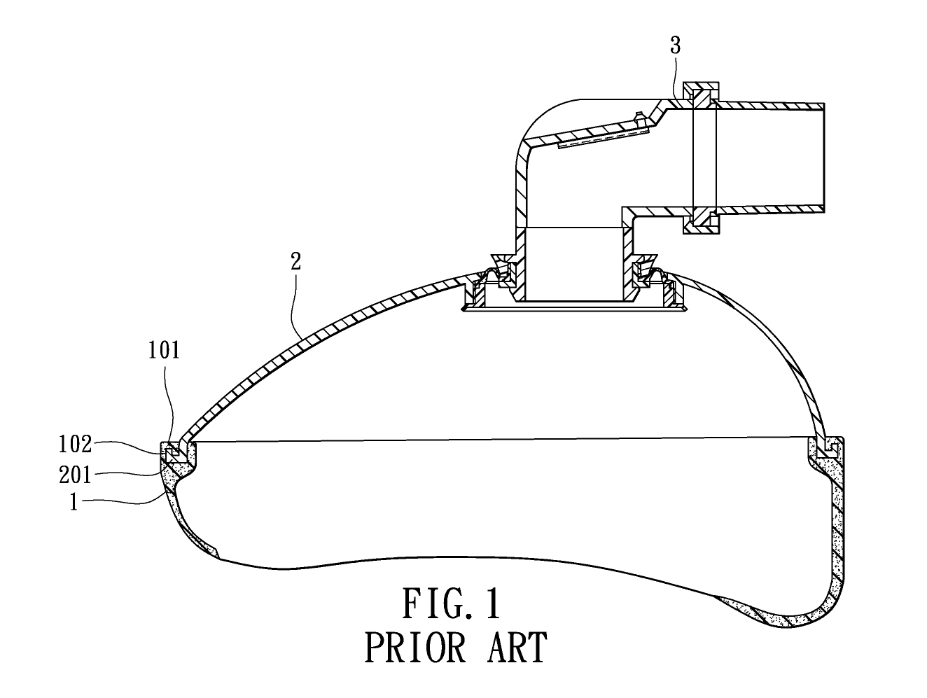

FIG. 2 is a partly sectional view of a respiratory mask according to the preferred embodiment of this invention;

FIG. 3 is a fragmentary enlarged sectional view of the preferred embodiment; and

FIG. 4 illustrates how the respiratory mask of the preferred embodiment is formed.

### DETAILED DESCRIPTION OF THE PREFERRED EMBODIMENT

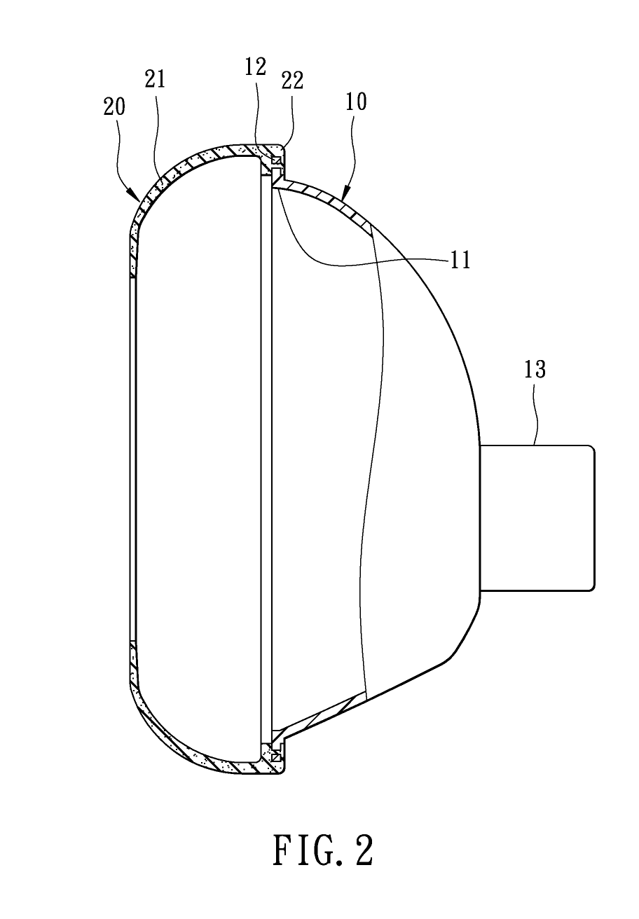

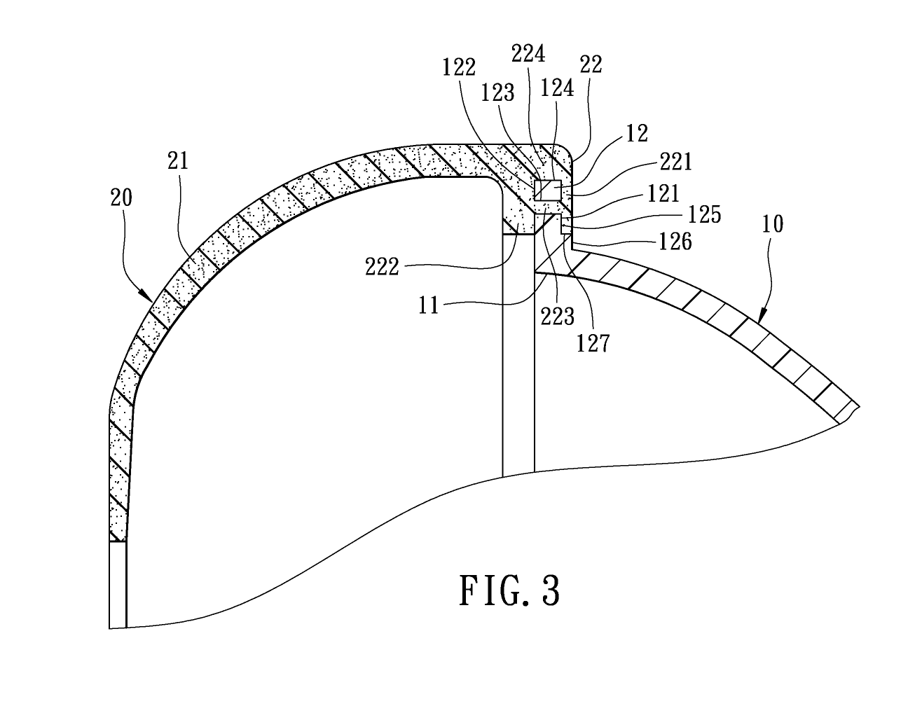

Referring to FIGS. 2 and 3, a respiratory mask according to the preferred embodiment of this invention comprises a cup-shaped body 10 and a hollow face-contact body 20.

The cup-shaped body 10 is made of polycarbonate (PC) in this embodiment and has an open end 11, an annular flange 12 extending outwardly from the open end 11, and a protruding portion 13 protruding from the cup-shaped body 10 opposite to the annular flange 12. The protruding portion 13 is to be connected to an air supply tube (not shown) for supplying oxygen into the respiratory mask. The annular flange 12 has a first annular surface 121, a second annular surface 122 opposite the first annular surface 121, a plurality of angularly spaced-apart through holes 123 extending through the first and second annular surfaces 121, 122, and an outer annular peripheral edge 124 interconnecting outer peripheries of the first and second annular surfaces 121, 122 and surrounding the through holes 123. In this embodiment, the first annular surface 121 is stepped and has a first step surface 125, a second step surface 126, and a shoulder portion 127 interconnecting the first and second step surfaces 125, 126. The through holes 123 are equidistantly formed in the first step surface 125 in this embodiment.

The hollow face-contact body 20 is made of silicone in this embodiment and is molded integrally to the cup-shaped body 10 by insert molding. The face-contact body 20 has a surrounding wall 21 and an annular connecting end portion 22 connected integrally as one piece with the surrounding wall 21. The annular connecting end portion 22 embeds the annular flange 12, and includes an outer annular plate 221 abutting against the first annular surface 121, an inner annular plate 222 abutting against the second annular surface 122, a plurality of connecting ribs 223 respectively filling the through holes 123 and interconnecting the outer and inner annular plates 221, 222, and an annular rim 224 surrounding the outer annular peripheral edge 124 and interconnecting outer ends of the outer and inner annular plates 221, 222. The outer annular plate 221 abuts against the first step surface 125. The outer annular plate 221 has the outer end opposite to the shoulder portion 127, and an inner end abutting against the shoulder portion 127. The inner annular plate 222 has a thickness greater than that of the outer annular plate 221. Preferably, a ratio of the thickness of the inner annular plate 222 to that of the outer annular plate 221 is not less than 2.5. In this embodiment, the ratio is approximately 3.

Referring back to FIG. 3, when the cup-shaped body 10 and the hollow face-contact body 20 are connected integrally as one piece, because the face-contact body 20 is made of silicone, the outer and inner annular plates 221, 222 respectively abut against the first and second annular surfaces 121, 122, the connecting ribs 223 interconnect the outer and inner annular plates 221, 222 and respectively fill the through holes 123, and the annular rim 224 surrounds the outer annular peripheral edge 124, the connection between the annular connecting end portion 22 and the annular flange 12 is strengthened.

The effects of this invention can be summarized as follows:

1. When high pressure air is supplied from the cup-shaped body 10, even if the high pressure air can exude between the second annular surface 122 and the inner annular plate 222, because the outer annular plate 221 and the inner annular plate 222 are interconnected through the connecting ribs 223 so that the outer annular plate 221 and the first annular surface 121 can abut tightly against each other, and because the connecting ribs 223 are respectively filled in the through holes 223, the high pressure air can be prevented from leaking through the connecting ribs 223 and the respective through holes 123.

2. Because the connecting ribs 223 interconnect the inner and outer annular plates 222, 221 and are filled in the respective through holes 123, the connection between the annular connecting end portion 22 and the annular flange 12 is strengthened. Even during cleaning of the respiratory mask, it is difficult to separate the cup-shaped body 10 and the face-contact body 20 from each other. Hence, the overall structure of the respiratory mask of the present invention is stable.

3. Because the thickness of the inner annular plate 222 is greater than that of the outer annular plate 221, and the ratio of the thickness of the inner annular plate 222 to that of the outer annular plate 221 is not less than 2.5, the inner annular plate 222 can be used to support positioning of the cup-shaped body 10.

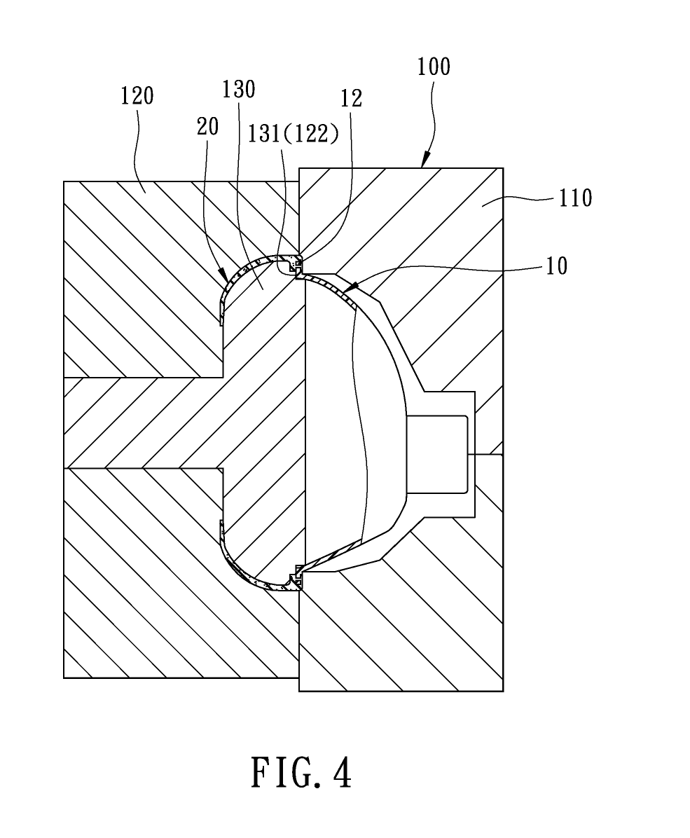

The respiratory mask of this embodiment is formed by molding using a mold 100, as shown in FIG. 4. The mold 100 has an upper mold 110 corresponding to the cup-shaped body 10, a lower mold 120 for forming the hollow face-contact body 20, and an inner mold 130. Through cooperation among the upper mold 110, the lower mold 120, and the inner mold 130, that is, after the pre-formed cup-shaped body 10 is placed in the mold 100, the face-contact body 20 can be connected directly to the annular flange 12 of the cup-shaped body 10. Further, through abutment of a mold surface 131 at a peripheral edge of the inner mold 130 against the second annular surface 122, overflow of silicone material for forming the face-contact body 20 can be prevented.

While the present invention has been described in connection with what is considered the most practical and preferred embodiment, it is understood that this invention is not limited to the disclosed embodiment but is intended to cover various arrangements included within the spirit and scope of the broadest interpretations and equivalent arrangements.

What is claimed is:

1. A respiratory mask, comprising:
a cup-shaped body having an open end and an annular flange extending outwardly from said open end, said annular flange having a first annular surface, a second annular surface opposite to said first annular surface, a plurality of angularly spaced-apart through holes extending through said first and second annular surfaces, and an outer annular peripheral edge interconnecting outer peripheries of said first and second annular surfaces and surrounding said through holes; and
a hollow face-contact body having a surrounding wall and an annular connecting end portion connected integrally as one piece with said surrounding wall, said annular connecting end portion embedding said annular flange and including an outer annular plate abutting against said first annular surface of said annular flange, an inner annular plate abutting against said second annular surface of said annular flange, a plurality of connecting ribs respectively filling said through holes and interconnecting said inner and outer annular plates, and an annular rim surrounding said outer annular peripheral edge of said annular flange and interconnecting outer ends of said inner and outer annular plates.

2. The respiratory mask of claim 1, wherein said first annular surface of said annular flange is stepped, and has a first step surface, a second step surface and a shoulder portion interconnecting said first and second step surfaces, said outer annular plate of said annular connecting end portion abutting against said first step surface, said outer end of said outer annular plate being opposite to said shoulder portion.

3. The respiratory mask of claim 2, wherein said outer annular plate of said annular connecting end portion has an inner end abutting against said shoulder portion.

4. The respiratory mask of claim 3, wherein said through holes in said cup-shaped body are equidistantly formed in said first step surface.

5. The respiratory mask of claim 1, wherein said inner annular plate of said annular connecting end portion has a thickness greater than that of said outer annular plate.

6. The respiratory mask of claim 5, wherein a ratio of the thickness of said inner annular plate to that of said outer annular plate is not less than 2.5.
